(19) United States
(12) Patent Application Publication
(10) Pub. No.: US 2025/0057548 A1
(43) Pub. Date: Feb. 20, 2025

(54) ASPIRATION CATHETER WITH FUNNEL TIP

(71) Applicant: Boston Scientific Scimed, Inc., Maple Grove, MN (US)

(72) Inventor: Cass Alexander Hanson, St. Paul, MN (US)

(73) Assignee: Boston Scientific Scimed, Inc., Maple Grove, MN (US)

(21) Appl. No.: 18/801,956

(22) Filed: Aug. 13, 2024

Related U.S. Application Data
(60) Provisional application No. 63/532,800, filed on Aug. 15, 2023.

(51) Int. Cl. A61B 17/22; A61B 17/00

(52) U.S. Cl. CPC A61B 17/22; A61B 17/00234; A61B 2017/00238; A61B 2017/00305; A61B 2017/00398; A61B 2017/00544; A61B 2017/00561; A61B 2017/22079; A61B 2217/005

(57) ABSTRACT

Example aspiration catheters and systems including aspiration catheters are disclosed. An example aspiration catheter includes a catheter body having a proximal end region, a distal end region and a lumen extending therein. The aspiration catheter also includes a fluid supply tube extending within the lumen of the catheter body, the fluid supply tube having a distal end region and a distal facing surface. The aspiration catheter also includes a funnel having a proximal end and a distal end, wherein the proximal end is coupled to the distal end region of the catheter body, and wherein the funnel is configured to shift between a collapsed configuration and an expanded configuration.

### CROSS-REFERENCE TO RELATED APPLICATIONS

This application claims the benefit of priority under 35 U.S.C. § 119 of U.S. Provisional Application No. 63/532,800, filed Aug. 15, 2023, the entire disclosure of which is hereby incorporated by reference.

### TECHNICAL FIELD

The disclosure is directed to aspiration systems. More particularly, the disclosure is directed to an aspiration catheter system for improved clot removal. In some instances, the aspiration catheter may include an expandable funnel tip.

### BACKGROUND

Thrombectomy is a procedure for removing thrombus from the vasculature of a patient. Mechanical and fluid-based systems can be used to remove thrombus. With fluid-based systems, an infusion fluid may be infused to a treatment area of a vessel with a catheter to dislodge the thrombus. In some instances, an effluent (e.g., the infusion fluid and/or blood) including the dislodged thrombus may be extracted from the vessel through the catheter. Of the known thrombectomy systems and methods, there is an ongoing need to provide alternative configurations of thrombectomy catheters and systems, as well as methods of operating such thrombectomy systems.

### SUMMARY

This disclosure provides design, material, manufacturing method, and use alternatives for medical devices. An example aspiration catheter includes a catheter body having a proximal end region, a distal end region and a lumen extending therein. The aspiration catheter also includes a fluid supply tube extending within the lumen of the catheter body, the fluid supply tube having a distal end region and a distal facing surface. The aspiration catheter also includes a funnel having a proximal end and a distal end, wherein the proximal end is coupled to the distal end region of the catheter body, and wherein the funnel is configured to shift between a collapsed configuration and an expanded configuration.

Alternatively or additionally to any of the examples above, wherein the fluid supply tube is configured to shift between a first position in which the distal end region is positioned proximal of the proximal end of the funnel and a second position in which the distal end region is positioned distal of the proximal end of the funnel.

Alternatively or additionally to any of the examples above, wherein the fluid supply tube is configured to shift between a first position in which the distal end region is positioned proximal of the proximal end of the funnel and a second position in which the distal end region is positioned between the distal end and the proximal end of the funnel.

Alternatively or additionally to any of the examples above, wherein the proximal end region of the catheter body is coupled to a fluid pump, and wherein the fluid pump is configured to cycle between applying a vacuum and ceasing to apply the vacuum, and wherein the fluid supply tube shifts between the first position and the second position based on the vacuum cycle of the fluid pump.

Alternatively or additionally to any of the examples above, wherein the fluid supply tube is configured to shift from a first position in which the distal end region is positioned proximal of the proximal end of the funnel and a second position in which the distal end region is positioned between the distal end and the proximal end of the funnel when the fluid pump is in a downstroke, and wherein the fluid supply tube is configured to shift from the second position to the first position when the fluid pump is in an upstroke.

Alternatively or additionally to any of the examples above, wherein when the fluid supply tube is in the second position, the distal facing surface is between 0 mm and 20 mm distal of the proximal end of the funnel.

Alternatively or additionally to any of the examples above, wherein when in the expanded configuration the funnel tapers from a first diameter at its proximal end to a second diameter at its distal end, and wherein the second diameter is greater than the first diameter.

Alternatively or additionally to any of the examples above, wherein the funnel includes a plurality of braided filaments.

Alternatively or additionally to any of the examples above, wherein funnel includes at least one reinforcing member, and wherein the at least one reinforcing member extends from a proximal end of the funnel to a distal end of the funnel.

Alternatively or additionally to any of the examples above, wherein the funnel is configured to direct thrombus into the lumen of the catheter shaft.

Alternatively or additionally to any of the examples above, wherein when in the expanded configuration the distal end region of the funnel is configured to engage an inner surface of a body vessel.

Alternatively or additionally to any of the examples above, further comprising at least one projection extending distally away from the distal facing surface of the fluid supply tube.

Alternatively or additionally to any of the examples above, wherein the fluid supply tube includes at least one distally projecting jet orifice for expelling at least one distally oriented fluid jet in a generally distal direction from the fluid supply tube within the catheter lumen or funnel.

Alternatively or additionally to any of the examples above, wherein the fluid supply tube includes at least one proximally projecting jet orifice for expelling at least one proximally oriented fluid jet in a generally proximal direction from the fluid supply tube within the catheter lumen or funnel.

Alternatively or additionally to any of the examples above, wherein the at least one distally projecting jet orifice is distal to the at least one proximally projecting jet orifice.

Alternatively or additionally to any of the examples above, wherein the at least one distally projecting jet orifice extends through a sidewall of the fluid supply tube.

Another example thrombectomy system includes a processor coupled to a fluid pump and a thrombectomy catheter, wherein the thrombectomy catheter includes a catheter body having a proximal end region, a distal end region and a lumen extending therein. The thrombectomy catheter also includes a fluid supply tube extending within the lumen of the catheter body, the fluid supply tube having a distal end region and a distal facing surface. The thrombectomy catheter also includes a funnel having a proximal end and a distal end, wherein the proximal end is coupled to the distal end region of the catheter body, and wherein the funnel is configured to shift between a collapsed configuration and an expanded configuration.

Alternatively or additionally to any of the examples above, wherein the fluid supply tube is configured to shift between a first position in which the distal end region is positioned proximal of the proximal end of the funnel and a second position in which the distal end region is positioned distal of the proximal end of the funnel.

Alternatively or additionally to any of the examples above, wherein the proximal end region of the catheter body is coupled to a fluid pump, and wherein the fluid pump is configured to cycle between applying a vacuum and ceasing to apply the vacuum, and wherein the fluid supply tube shifts between the first position and the second position based on the vacuum cycle of the fluid pump.

Another example thrombectomy system includes a processor coupled to a fluid pump and a thrombectomy catheter, wherein the thrombectomy catheter includes a catheter body having a proximal end region, a distal end region and a lumen extending therein. The thrombectomy catheter also includes a fluid supply tube extending within the lumen of the catheter body, the fluid supply tube having a distal end region and a distal facing surface. The thrombectomy catheter also includes a funnel having a proximal end and a distal end, wherein the proximal end is coupled to the distal end region of the catheter body, and wherein the funnel is configured to shift between a collapsed configuration and an expanded configuration. Further, the fluid supply tube is configured to shift between a first position in which the distal end region is positioned proximal of the proximal end of the funnel and a second position in which the distal end region is positioned distal of the proximal end of the funnel. Further, the fluid pump is configured to cycle between applying a vacuum and ceasing to apply the vacuum, and wherein the processor is configured to direct the fluid supply tube to shift between the first position and the second position based on the vacuum cycle of the fluid pump.

The above summary of some example embodiments is not intended to describe each disclosed embodiment or every implementation of the disclosure.

### BRIEF DESCRIPTION OF THE DRAWINGS

The disclosure may be more completely understood in consideration of the following detailed description of various embodiments in connection with the accompanying drawings, in which:

FIG. 1 is a perspective view of an illustrative thrombectomy system;

FIG. 2 is a partially exploded perspective view of a portion of the thrombectomy system of FIG. 1;

FIG. 3 is a partially exploded side view of a portion of the thrombectomy system shown in FIG. 1;

FIG. 4 is a perspective view of a distal end region of an illustrative thrombectomy catheter;

FIG. 5 is a cross-sectional view of a distal end region of the thrombectomy catheter shown in FIG. 4;

FIG. 6 is a cross-sectional view of a distal end region of the thrombectomy catheter shown in FIG. 4;

FIG. 7 is a cross-sectional view of a distal end region of another embodiment of the thrombectomy catheter shown in FIG. 4;

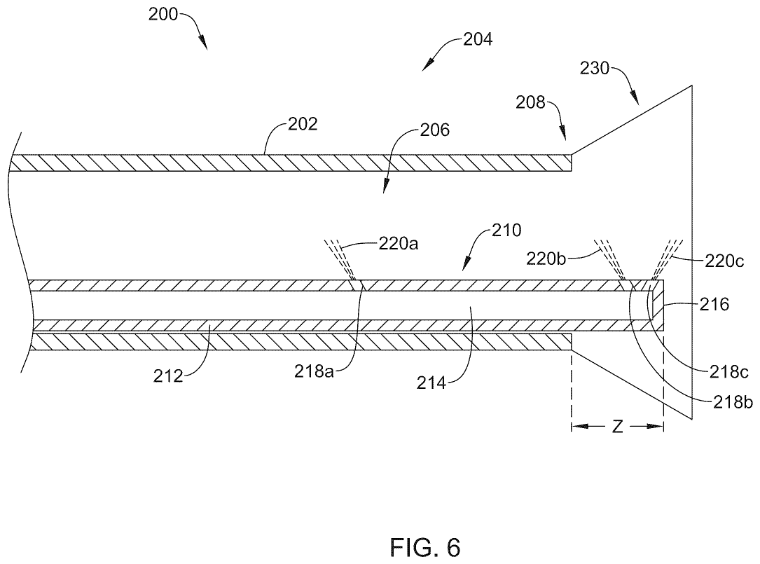

FIG. 6 is a longitudinal cross-sectional view of a distal end region of another illustrative thrombectomy catheter;

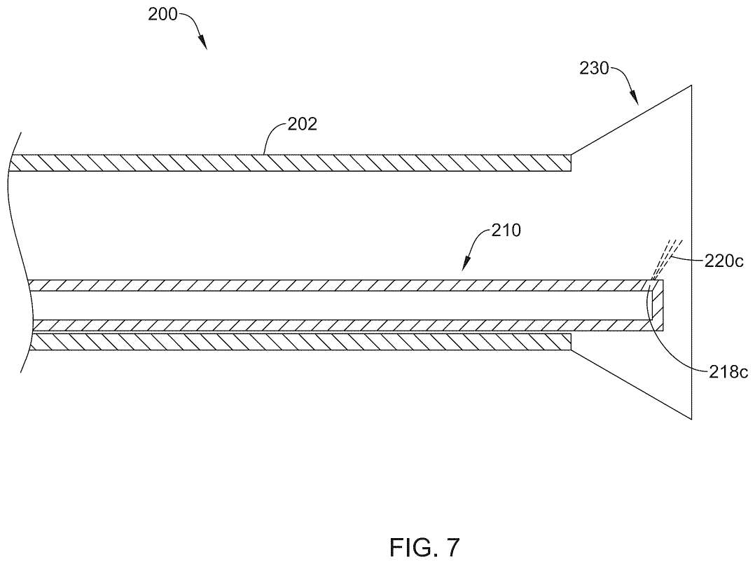

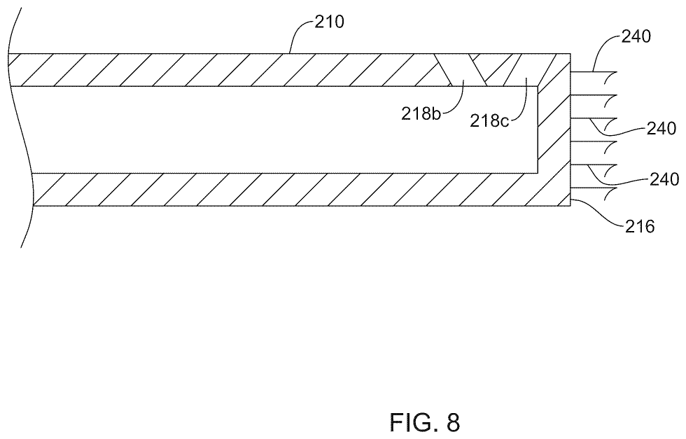

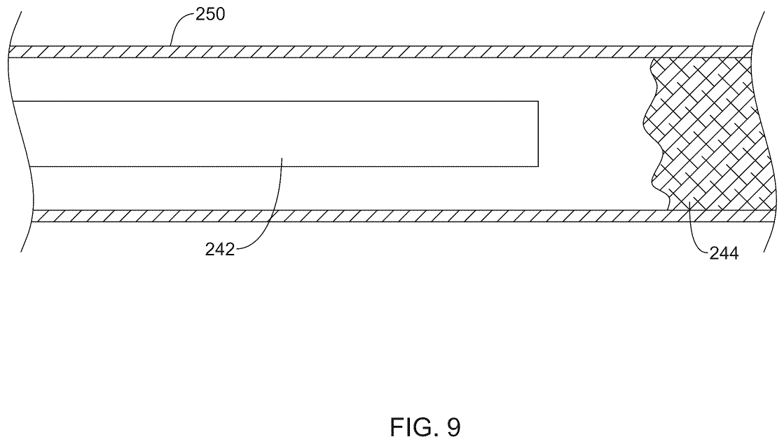

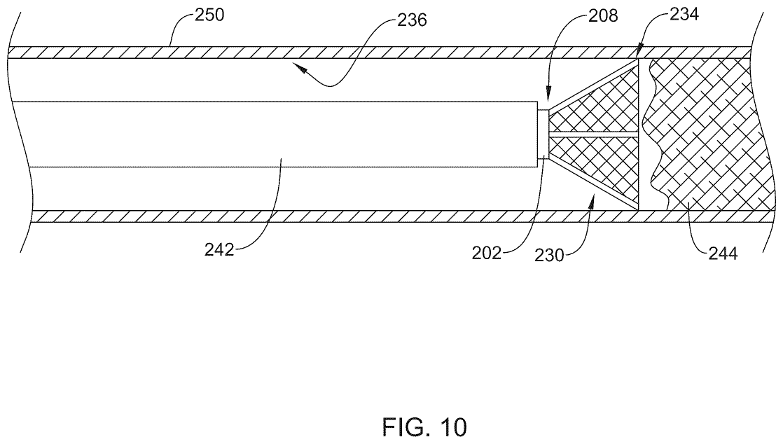

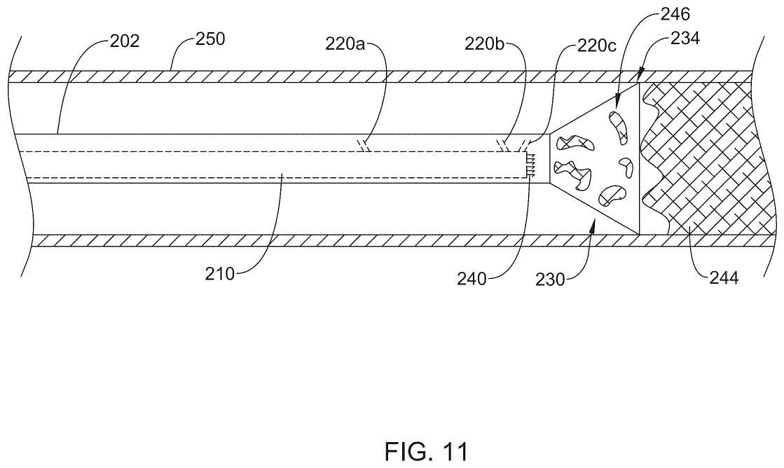

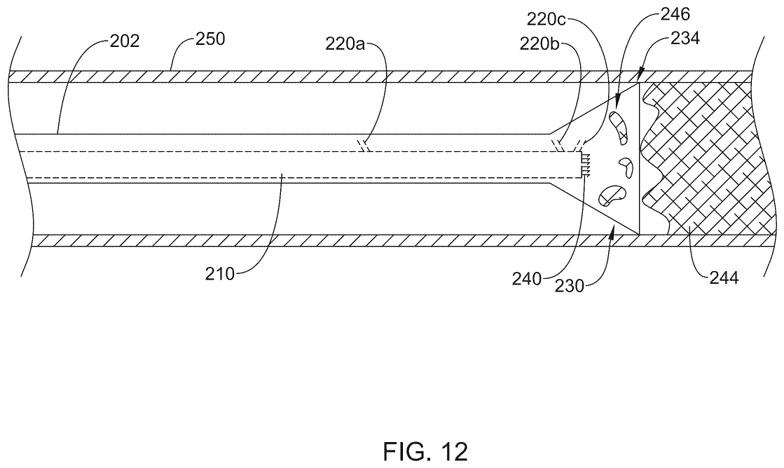

FIG. 7 is a longitudinal cross-sectional view of a distal end region of another illustrative thrombectomy catheter;

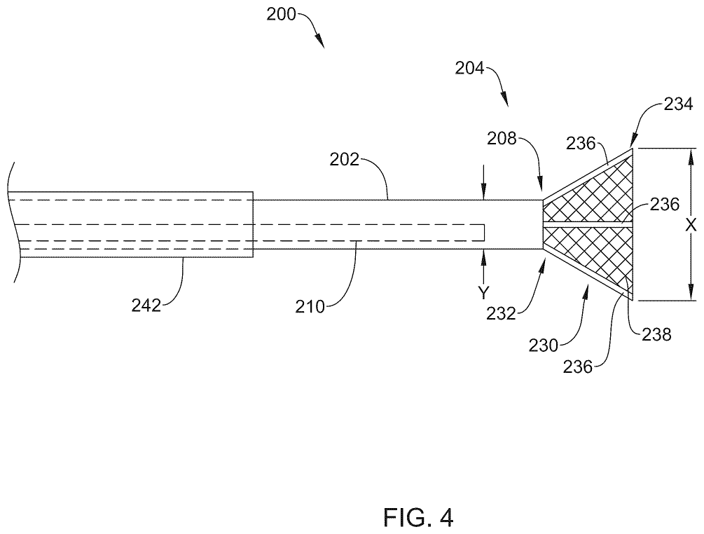

FIG. 8 is a side view of a portion of the thrombectomy catheter shown in FIG. 4;

FIGS. 9-12 illustrate an example thrombectomy catheter being utilized to treat a thrombus inside a body vessel.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

All numeric values are herein assumed to be modified by the term “about”, whether or not explicitly indicated. The term “about” generally refers to a range of numbers that one of skill in the art would consider equivalent to the recited value (e.g., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

Thrombectomy catheters and systems may be used to remove thrombus, plaques, lesions, clots, etc. from veins or arteries. Some thrombectomy catheters may utilize high velocity saline jets in a series to entrain fluid or clot material into and through the shaft of the catheter. Other thrombectomy systems may utilize one or more pressurized saline jets which travel backwards to create a low-pressure zone and a vacuum effect, whereby the vacuum pulls clot material into and through the shaft of the catheter. However, prolonged operation of a thrombectomy system may result in the detrimental removal of excess blood from the veins or arteries of a patient. Accordingly, it may be desirable to design a thrombectomy system which operates (e.g., turns on to remove thrombus, plaque, blood, etc.) only when the distal tip of the system senses the presence of clot material. Thrombectomy systems which operate (e.g., turn on to remove thrombus, plaque, blood, etc.) only when the distal tip of the system senses the presence of clot material are disclosed herein.

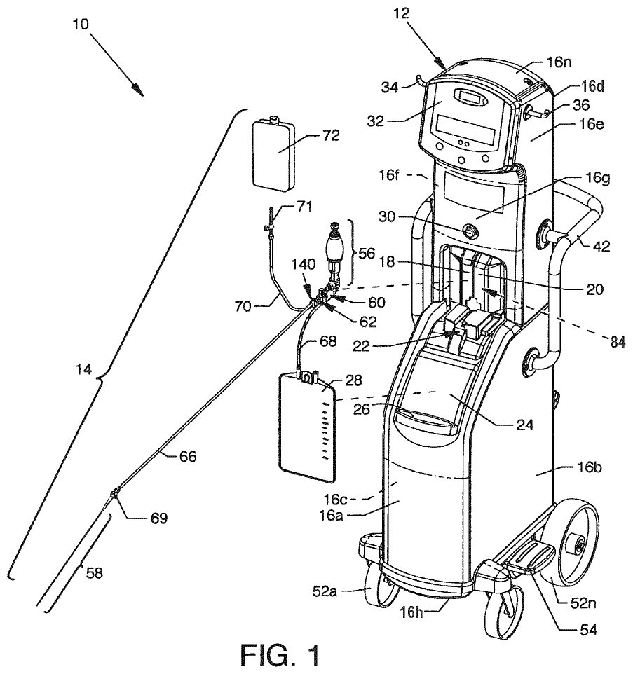

FIG. 1 is a perspective view of an illustrative thrombectomy system 10. The thrombectomy system 10 may include a control console or drive unit 12 and a pump/catheter assembly 14. In some instances, the pump/catheter assembly 14 may be a single use device in which a new pump/catheter assembly 14 may be used with the drive unit 12 for each medical procedure. Shown on the drive unit 12 are a plurality of removable panels 16a-16n about and along the drive unit 12 enclosing the internal structure of the drive unit 12. An illustrative drive unit 12 is described in commonly assigned U.S. Pat. No. 7,935,077, titled THROMBECTOMY CATHETER DEPLOYMENT SYSTEM, the disclosure of which is hereby incorporated by reference. Centrally located in the drive unit 12 and aligned to the lower region of the panel 16g may be automatically opening doors 18 and 20 which open to expose the interior of the drive unit 12 to provide access to a carriage assembly 22. The carriage assembly 22, which may accommodate components of the pump/catheter assembly 14, as discussed further herein, is shown accessible via opening the closed doors 18 and 20. The drive unit 12 may include a catch basin for collecting fluid leakage from the components of the pump/catheter assembly 14. For example, a removable drip tray 24 is shown located on the front of the drive unit 12 extending from below the carriage assembly 22 toward the panel 16a. Other configurations of catch basins are also contemplated. The drip tray 24 and a removable receptacle 26 may collectively support and accommodate an effluent collection bag, such as effluent collection bag 28 of the pump/catheter assembly 14. In other instances, the drive unit 12 may include a different structure, such as a hook for hanging the effluent collection bag 28 from, or a shelf for setting the effluent collection bag 28 on. In instances where the carriage assembly 22 is movable, a carriage assembly activation switch 30 may be provided with the drive unit 12, such as located on panel 16g, to selectively position the carriage assembly 22 inwardly or outwardly. A user interface 32, including memory capabilities, may be provided with the drive unit 12, such as located at the upper region of the drive unit 12 between the upper regions of the upper side panels 16e and 16f. Saline bag hooks 34 and 36 may extend through the panels 16e and 16f to hang saline bags therefrom. The drive unit 12 may include a handle 42 as well as a plurality of wheels 52a-52n and brake pedals 54 for wheel lockage to assist in maneuvering the drive unit 12 by medical personnel.

The pump/catheter assembly 14, which may be a disposable single-use device, is shown unattached from the drive unit 12. The pump/catheter assembly 14 includes a pump 56 and a thrombectomy catheter 58. During use, a portion of the pump/catheter assembly 14 may be secured within a portion of the drive unit 12. Other components included in the pump/catheter assembly 14 may include a bubble trap 60 attached to the pump 56, a connection manifold assembly 62 connected to the bubble trap 60, an effluent return tube 66 connected between the connection manifold assembly 62 and the thrombectomy catheter 58, a high-pressure fluid supply tube 64 attached between the output of the pump 56 and the thrombectomy catheter 58 which may be coaxially arranged inside the effluent return tube 66, a transition fixture 69 between the distal end of the effluent return tube 66 and the proximal end of the thrombectomy catheter 58, an effluent waste tube 68 connecting the effluent collection bag 28 to the connection manifold assembly 62, and a fluid supply tube 70 having a bag spike 71 connecting a fluid supply bag 72 (e.g., a saline bag) to the connection manifold assembly 62. The fluid supply tube 70 may be in fluid communication with the interior of the bubble trap 60 to provide fluid from the fluid supply bag 72 to the pump 56 and then to the thrombectomy catheter 58 through the high-pressure fluid supply tube 64.

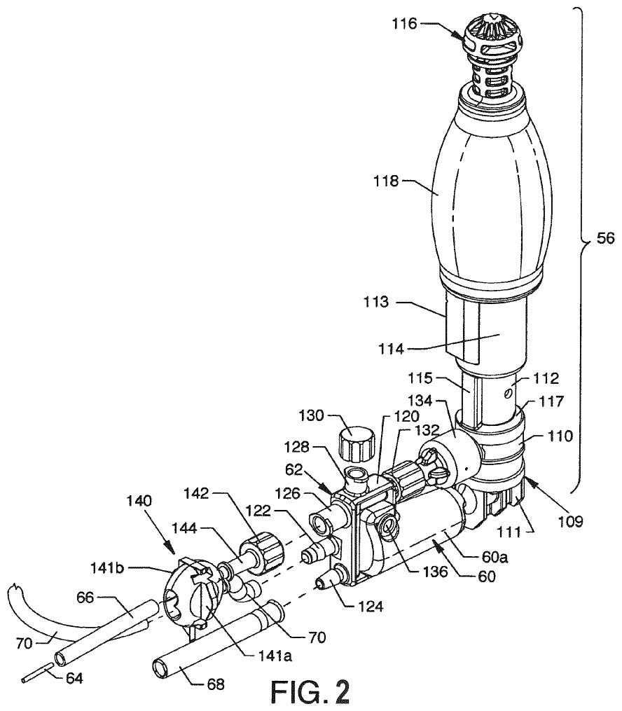

FIG. 2 is a partially exploded perspective view of several components of the pump/catheter assembly 14 generally including the pump 56, the bubble trap 60, the connection manifold assembly 62, and a fixture 140. The pump 56 centers about a tubular body 112. Components are located about the lower region of the tubular body 112 and include a base 109 having an upper portion 110 and a lower portion 111 both positioned about the lower region of the tubular body 112. An annular surface 117 is included at the top of the upper portion 110 of the base 109 for intimate contact with capture tabs of the carriage assembly 22 to contain the pump 56 within the carriage assembly 22. A top body 114, is positioned about the upper region of the tubular body 112. The base 109 and the top body 114, as well as a connecting panel 115, may be molded or otherwise suitably constructed to encompass the greater part of the tubular body 112, for example. A data plate 113 may also be included on the top body 114 for the inclusion of a barcode, an RFID tag, or other informational displays to determine operational parameters of the device.

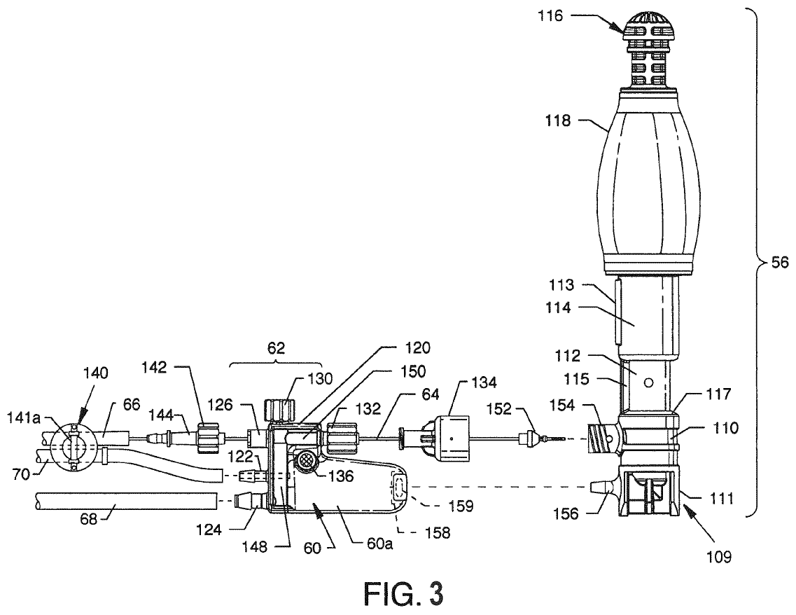

The pump 56 may include a hemispherically-shaped pump piston head 116 having a flexible boot 118 connected to and extending between the top body 114 and the pump piston head 116. In some instances, the geometrically configured lower portion 111 of the base 109 may serve as a mount for one end of the bubble trap 60 (FIG. 3).

The connection manifold assembly 62 may be secured directly to the other end of the bubble trap 60 and in some instances may include a bracket 120 to which is attached a vertically oriented tubular manifold 148 having a plurality of ports attached or formed therethrough including a fluid (e.g., saline) inlet port 122, an effluent outlet port 124, a Luer style effluent return port 126, and/or an auxiliary port 128 and cap 130. Also shown are connectors 132 and 134 connectingly extending between the connection manifold assembly 62 and the upper portion 110 of the base 109.

The bubble trap 60 may include mating halves of which one mating half 60a is shown. A hydrophobic filter 136 may be included at the upper forward region of the bubble trap half 60a. Another hydrophobic filter may be included on the second bubble trap half (not explicitly shown) which opposes the hydrophobic filter 136 on the bubble trap half 60a.

The fixture 140, and components associated therewith, assists in support and connection of the effluent return tube 66 to the effluent return port 126 by a connector 142 combined continuously with a connection tube 144, and also assists in support, passage and connection of the fluid supply tube 70 with the fluid inlet port 122. The fixture 140 may include outwardly extending vertically aligned and opposed tabs 141a and 141b which prevent the fixture 140 and associated effluent return tube 66 containing the high-pressure fluid supply tube 64 and the fluid supply tube 70 from contacting a roller pump (not explicitly shown) provided with the drive unit 12, such as located in the carriage assembly 22 or adjacent thereto.

FIG. 3 is a partially exploded side view of the elements of FIG. 2 illustrating the relationship of the pump 56, the bubble trap 60, the connection manifold assembly 62, and the fixture 140. Also shown is the vertically oriented tubular manifold 148 secured to the bracket 120. The effluent outlet port 124 may be connected to and in fluid communication with the lower interior of the tubular manifold 148. The effluent return port 126 may be connected to and in fluid communication with the upper interior of the tubular manifold 148. Also connecting to the tubular manifold 148 is a horizontally aligned passage port 150 and associated connector 132, each opposing the effluent return port 126. The passage port 150 may accommodate the high-pressure fluid supply tube 64 which extends distally through the lumen (not explicitly shown) of the passage port 150, the connector 132, the upper region of the tubular manifold 148, the effluent return port 126, the connector 142, the connection tube 144, and into and through the effluent return tube 66 to connect to the thrombectomy catheter 58 (FIG. 1). The proximal end of the high-pressure fluid supply tube 64 includes a high-pressure fitting 152 located near the proximal end of the high-pressure fluid supply tube 64 to facilitate connection of the high-pressure fluid supply tube 64 in fluid communication with the interior of the pump 56. The proximal end of the high-pressure fluid supply tube 64, which is the inlet to the high-pressure fluid supply tube 64, may include a plurality of very small holes (not shown) comprising a filter at the proximal end thereof. The connector 134, which may have internal and/or external threads, may be aligned over and about the high-pressure fluid supply tube 64 distal to the high-pressure fitting 152 and threadingly engage a threaded connection port 154 extending horizontally from the upper portion 110 of the base 109 of the pump 56. The connector 134 may be rotated to threadably engage the high-pressure fitting 152 with a corresponding mating threaded structure provided with the pump 56. A connector 132 may be utilized to engage the externally threaded end of the connector 134 to secure the connector 134, and thus the pump 56, to the connection manifold assembly 62 and to provide for fixation of the bubble trap 60 to the pump 56. In addition, direct connection and fluid communication between the pump 56 and the bubble trap 60 may be provided by a horizontally oriented pump fluid inlet port 156 which engages a corresponding receptor port 158 and seal 159 interior to one end of the bubble trap 60. The fluid inlet port 122 located on the bracket 120 may extend behind the tubular manifold 148 to communicate with the interior of the bubble trap 60 for fluid (e.g., saline) debubbling, whereby unpressurized fluid (e.g., saline) is made available for use by the pump 56.

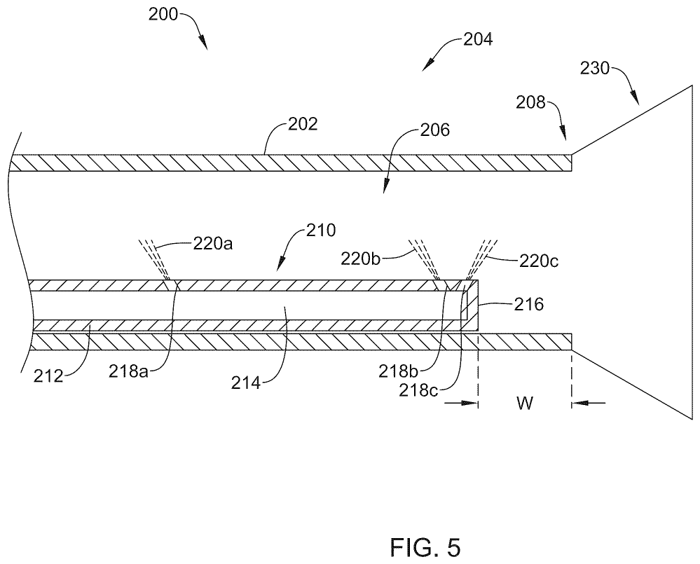

FIG. 4 is a perspective view of a distal end region 204 of an illustrative thrombectomy catheter 200. The thrombectomy catheter 200 may be one illustrative example of the thrombectomy catheter 58 described above. The thrombectomy catheter 200 may include a tubular member or catheter body 202 extending from a proximal end region (not explicitly shown) configured to remain outside the patient to a distal end region 204. The catheter body 202 may include a distal end 208. The catheter body 202 may be one illustrative example of, or be in fluid communication with, the effluent return tube 66 of the thrombectomy catheter 58 described above. A lumen 206 (shown in FIG. 5) may extend from the proximal end region to the distal end region 204 of the catheter body 202. While not explicitly shown, the catheter body 202 may include one or more markers (e.g., radiopaque marker bands) disposed along the catheter body 202. Further, while not explicitly shown, in some embodiments, the catheter body 202 may include one or more openings extending through a sidewall thereof, if desired.

The thrombectomy catheter 200 may further include a high-pressure fluid supply tube 210. The high-pressure fluid supply tube 210 may be one illustrative example of, or be in fluid communication with, the high-pressure fluid supply tube 66 of the thrombectomy catheter 58 described above. The high-pressure fluid supply tube 210 may be disposed within and extend through the lumen 206 of the catheter body 202.

FIG. 4 further illustrates that the thrombectomy catheter 200 may further include a funnel 230. The funnel 230 may include a proximal end 232 and a distal end 234. The proximal end 232 of the funnel 230 may be attached to the distal end 208 of the catheter body 202.

In some examples, the funnel 230 may be a self-expandable funnel configured to shift between a first configuration in which the funnel 230 is in an unexpanded configuration and a second configuration in which the funnel 230 is in an expanded configuration. For example, FIG. 4 illustrates that the thrombectomy catheter 200 may be delivered to a treatment site through a tubular delivery shaft 242. The funnel 230 may be collapsed and loaded into the lumen of the delivery shaft 242. Once loaded in the lumen of the delivery shaft 242, the delivery shaft may track through a patient's body vessel adjacent to a target treatment site (e.g., thrombus). After being positioned adjacent to the target treatment site, the delivery shaft 242 may be retracted in a distal-to-proximal direction. It can be appreciated that upon proximal retraction of the delivery shaft 242, the self-expanding funnel 230 may shift from the unemployed, delivery configuration to the deployed configuration. Further, in some examples, when in the deployed configuration, the funnel 230 may be designed to expand such that the distal end of the funnel 230 engages the inner surface of a body vessel within which it is deployed.

The funnel 230 may generally be conically shaped in the expanded configuration. For example, FIG. 4 illustrates that when expanded, the outer diameter of the proximal end 232 of the funnel 230 may match the outer diameter “Y” of the distal end 208 of the catheter body 202 (as the proximal end 232 of the funnel 230 is attached to the distal end 208 of the catheter body 230). Further, FIG. 4 illustrates that the funnel 230 may taper outwardly to a diameter “X”. FIG. 4 illustrates that the diameter X may be larger than the diameter Y. In some examples, the outer diameter of the distal end 234 of the funnel 230 may be about 2 mm to about 18 mm, or about 4 mm to about 15 mm, or about 6 mm to about 14 mm, about 8 mm to about 12 mm.

As discussed herein, the funnel 230 may be a self-expanding funnel 230. The self-expanding funnel 230 may include one or more struts 238 combined to form a rigid and/or semi-rigid stent structure. For example, the funnel struts 238 may be wires or filaments which are braided, wrapped, intertwined, interwoven, weaved, knitted, looped (e.g., bobbinet-style) or the like to form the funnel 230 structure. Alternatively, the funnel 230 may be a monolithic structure formed from a cylindrical tubular member, such as a single, cylindrical tubular laser-cut Nitinol tubular member, in which the remaining portions of the tubular member form the funnel struts 238. Openings or interstices through the wall of the funnel 238 may be defined between adjacent funnel struts 238.

In other examples, the funnel 230 may be balloon expandable. For example, a balloon expandable funnel 230 may be advanced to a target treatment site in an unexpanded, delivery configuration whereby a balloon is positioned within the funnel 230 and inflated to deploy the funnel 230 from the unexpanded configuration to a deployed, expanded configuration.

The funnel 230 in examples disclosed herein may be constructed from a variety of materials. For example, the funnel 230 (e.g., self-expanding or balloon expandable) may be constructed from a metal (e.g., Nitinol, Elgiloy, etc.). In other instances, the funnel 230 may be constructed from a polymeric material (e.g., PET). In yet other instances, the funnel 230 may be constructed from a combination of metallic and polymeric materials. Additionally, the funnel 230 may include a bioabsorbable and/or biodegradable material.

FIG. 5 is a schematic cross-sectional view of a distal end region 204 of the thrombectomy catheter 200 illustrated in FIG. 4. The thrombectomy catheter 200 may be one illustrative example of the thrombectomy catheter 58 described above. The thrombectomy catheter 200 may include a tubular member or catheter body 202 extending from a proximal end region (not explicitly shown) configured to remain outside the body to a distal end region 204. FIG. 5 illustrates the funnel 230 extending distally from a distal end of the catheter body 202. In FIG. 5, the funnel 230 is shown in an expanded configuration.

The catheter body 202 may be one illustrative example of, or be in fluid communication with, the effluent return tube 66 of the thrombectomy catheter 58 described above. A lumen 206 may extend from the proximal end region to the distal end 208 of the catheter body 202. It can be appreciated that the funnel 230 may be designed to direct thrombus or other body material into the lumen 206. In some instances, the distal opening of the funnel 230 may be in a plane that extends generally orthogonal to a longitudinal axis of the catheter body 202. In other instances, the distal opening of the funnel 230 may be in a plane that extends generally oblique to a longitudinal axis of the catheter body 202. Generally, the distal opening of the funnel 230 may be an entrainment inflow orifice (e.g., opening). While not explicitly shown, the catheter body 202 may include one or more markers (e.g., radiopaque marker bands) disposed along the catheter body 202 and/or funnel 230. Further, while not explicitly shown, in some embodiments, the catheter body 202 may include one or more openings extending through a sidewall thereof, if desired.

As discussed herein, the thrombectomy catheter 200 may further include a high-pressure fluid supply tube 210. The high-pressure fluid supply tube 210 may be one illustrative example of, or be in fluid communication with, the high-pressure fluid supply tube 66 of the thrombectomy catheter 58 described above. The high-pressure fluid supply tube 210 may be disposed within and extend through the lumen 206 of the catheter body 202. The high-pressure fluid supply tube 210 may include a supply tube wall 212 defining a lumen or fluid pathway 214 extending therethrough. In at least some instances, the high-pressure fluid supply tube 210 may have a closed distal end 216. Because of this, fluid may be able to pass distally through the fluid pathway 214 but does not exit the distal end. The high-pressure fluid supply tube 210 may extend along a length of the catheter body 202 with the distal end 216 located within the lumen 206 of the catheter body 202 proximal to the distal end 208 of the catheter body 202. A proximal end of the high-pressure fluid supply tube 210 may be in fluid communication with the pump 56 described herein, to provide high-pressure fluid to the fluid pathway 214 of the high-pressure fluid supply tube 210.

A plurality of jet orifices 218a-c (collectively, 218) may be defined along the supply tube wall 212. For example, the supply tube wall 212 may include two, three, four, five, six, or more jet orifices 218. The jet orifices 218 may be spaced along the supply tube wall 212 at any desired intervals. For example, each of the jet orifices 218 may be equidistantly spaced from adjacent jet orifices 218 along the length of the supply tube wall 212. In other instances, the jet orifices 218 may be arranged such that the spacing between adjacent jet orifices 218 near the distal end of the supply tube wall 212 is closer than the spacing between adjacent jet orifices 218 near the proximal end of the supply tube wall 212. For instance, the spacing between the orifices 218 may gradually increase as you move proximally along the length of the shaft, or the spacing may increase in a stepwise configuration. In some instances, some or all of the jet orifices 218 may be axially aligned along the supply tube wall 212. In other instances, one or more of the jet orifices 218 may be circumferentially offset from one another about the supply tube wall 212. A number of patterns are contemplated including a helical pattern, a pattern where no two jet orifices 218 are disposed at the same axial location, a regular pattern including two or more jet orifices 218 disposed at the same axial location, an irregular pattern (where some of the jet orifices 218 may or may not be disposed at the same axial location), etc.

The jet orifices 218 may be formed using a suitable method such as electron discharge machining, etching, cutting (e.g., including laser cutting), or the like. In some instances, one or more of the jet orifices 218 may have a substantially round shape. In other instances, one or more of the jet orifices 218 may have a substantially non-round shape (e.g., oval, polygonal, irregular, etc.). In some instances, the jet orifices 218 may be beveled or otherwise include a beveled surface. It is contemplated that a size and/or a shape of the jet orifices 218 may be varied to vary the velocity of the fluid exiting the jet orifices. For example, decreasing the size of the jet orifices 218 may increase the velocity of the fluid exiting the jet orifices 218. In some embodiments, the size of the jet orifices 218 may be varied based on the pressure capacity of the thrombectomy system, the number of jet orifices, the dimensions of the high-pressure fluid supply tube 210 (e.g., length, wall thickness, inner diameter, etc.), and/or combinations thereof. In some examples, the jet orifices 218 may have a cross-sectional dimension in the range of about 0.0005″ to about 0.0030″, or about 0.0010″ to about 0.0025″, or about 0.0015″ to about 0.0020″.

Infusion of motive fluid through the lumen 214 of the supply tube wall 212 may result in fluid being jetted through the jet orifices 218 and the generation of a proximally directed aspiration force. At least some of the jet orifices 218a-b may be angled in a proximal direction or otherwise designed to infuse fluid (e.g., a motive fluid, a liquid, a gas or air, steam, a fluid with particles disposed therein, or the like) through the jet orifices 218a-b and into the lumen 206 of the catheter body 202 in a generally proximal direction as depicted by lines 220a-b representing motive jetted fluid projecting generally proximally from the jet orifices 218a-b. For example, each of the jet orifices 218a-b may be arranged at an acute angle to the longitudinal axis of the supply tube wall 212 such that the jet orifices 218a-b angle in a proximal direction.

In some embodiments, one or more of the jet orifices 218c may be designed to infuse fluid (e.g., a motive fluid, a liquid, a gas or air, steam, a fluid with particles disposed therein, or the like) through the jet orifice(s) 218c and into the lumen 206 of the catheter body 202 and/or the funnel 230 in a generally distal direction as depicted by lines 220c representing motive jetted fluid projecting generally distally from the jet orifice 218c. For example, the jet orifice 218c may be arranged at an oblique angle to the longitudinal axis of the supply tube wall 212 such that the jet orifice 218c angles in a distal direction. It is contemplated that an angle of the jet orifices 218 and thus the motive jetted fluid 220 may be varied to adjust the velocity of the fluid exiting the jet orifices 218. As further described herein, the supply tube wall 212 may include one or more, or a plurality of proximally oriented or directed jet orifices 218a, 218b (i.e., jet orifices configured to direct fluid infused through the lumen 214 of the supply tube wall 212 in a proximal direction) and the supply tube wall 212 may include one or more, or a plurality of distally oriented or directed jet orifices 218c (i.e., jet orifices configured to direct fluid infused through the lumen 214 of the supply tube wall 212 in a distal direction). In some examples, the distally projecting jet orifice 218c may be axially aligned with one or more of the proximally projecting jet orifices 218a-b. In other examples, the distally projecting jet orifice 218c may be circumferentially offset from one or more of the proximally projecting jet orifices 218a-b. For example, the distally projecting jet orifice 218c may be circumferentially offset from one or more of the proximally projecting jet orifices 218a-b by in the range of about 100 to about 350° or about 450 to about 135°.

The distally projecting jet orifice 218c may be the distalmost jet orifice, with the proximally projecting jet orifices 218a-b positioned proximal of the distally projecting jet orifice 218c. However, this is not required. In some embodiments, the distally projecting jet orifice 218c may be positioned proximal to at least one proximally projecting jet orifice 218a-b. While the supply tube wall 212 is illustrated as including only a single distally projecting jet orifice 218c, the supply tube wall 212 may include more than one distally projecting jet orifice, as desired. When more than one distally projecting jet orifice 218c is provided, the distally projecting jet orifices may be positioned at differing axial and/or circumferential locations from one another or similar axial and/or circumferential locations as one another, as desired. The distally projecting jet orifice(s) 218c may break up particles as they are drawn into the lumen 206 of the catheter body 202 while the proximally projecting jet orifices 218a-b may move particles proximally along the catheter body 202.

The performance of the thrombectomy catheter 200 and the high-pressure fluid supply tube 210 may be directly related to the velocity of the motive jetted fluid 220 exiting the jet orifices 218 and the shear-induced turbulent flux created by the jetted motive fluid 220. For example, the more powerful the jetted motive fluid 220, the higher the aspiration rates may be. It is further contemplated that the performance of the jet-powered aspiration catheter 200 may be directly related to the speed at which the clot can be entrained into the catheter 200, macerated, and removed from the body. Any clogging that occurs within the catheter body 202 may reduce or completely stop the removal of the clot.

In some configurations, the distally projecting jet orifice 218c may be proximally spaced a distance “W” from the distal end 208 of the catheter body 202. It is contemplated that the longitudinal location of the distally projecting jet orifice 218c on the supply tube wall 212 and relative to the distal end 208 of the catheter body 202 may be varied based on a size of the aperture of the distally projecting jet orifice 218c, the velocity of the fluid within the lumen 214 of the supply tube wall 212, the angle of the distally projecting jet orifice 218c, or combinations thereof, etc. to ensure the distally oriented motive jetted fluid 220c impinges the inner surface of the catheter body 202. In some examples, the distance W that the distally projecting jet orifice 218c may be proximally spaced from the distal end 208 of the catheter body 202 may be about 0 mm to about 10 mm, or about 2 mm to about 8 mm, or about 4 mm to about 6 mm, or about 5 mm.

In some instances, the jet orifices 218 may be oriented at an angle relative to the longitudinal axis of the supply tube wall 212. For example, the proximally projecting jet orifices 218a-b may be oriented at an oblique (e.g., acute) angle relative to the longitudinal axis of the supply tube wall 212 and/or oriented at an angle greater than zero degrees and less than ninety degrees relative to the longitudinal axis of the supply tube wall 212. It is contemplated that a distally projecting jet orifice 218c may be oriented at an oblique (e.g., obtuse) angle relative to the longitudinal axis of the supply tube wall 212 and/or oriented at an angle greater than 90 degrees and less than 180 degrees relative to the longitudinal axis of the supply tube wall 212. In other instances, the jet orifices 218 may be oriented perpendicular to the longitudinal axis of the supply tube wall 212 (e.g., at an angle of about 90 degrees relative to the longitudinal axis of the supply tube wall 212). The angle may or may not be the same for all the jet orifices 218.

In at least some instances, the jet orifices 218 may be understood as being arranged in series. In other words, the jet orifices 218 may be arranged such that adjacent jet orifices 218 are spaced longitudinally apart at various locations along the longitudinal axis of the supply tube wall 212. For example, the jet orifices 218 may be uniformly or non-uniformly spaced along of a length of the supply tube wall 212. This may position the jet orifices 218 at axially spaced apart locations within the catheter body 202 and along the length thereof. For example, the jet orifices 218 may be spaced along an entire length of the supply tube wall 212 and correspondingly along an entire length of the catheter body 202, or portions thereof, as desired. In some examples, the jet orifices 218 may be spaced at intervals in the range of every 5 inches (12.7 centimeters (cm)) to every 15 inches (38.1 cm), or in the range of every 6 inches (15.2 cm) to every 12 inches (30.5 cm) along a length of the supply tube wall 212. In other instances, the spacing between the jet orifices 218 may be less than every 5 inches (12.7 cm) or greater than every 15 inches (38.1 cm). Accordingly, motive fluid leaves via the jet orifices 218 forming a jetted motive fluid 220a-c (collectively, 220).

In some instances, entrainment material may enter the distal opening of the funnel 230 and then may be urged proximally by momentum transfer. As the mixture of the jetted motive fluid 220 and entrainment material migrates proximally, the material may sequentially approach a number of the jet orifices 218. Upon interaction with the jetted motive fluid 220 from each individual jet orifice 218, the momentum in the entrainment material mixture may increase, and the thrombogenic material may more readily flow proximally through the catheter body 202 for removal. The increase in momentum may allow for the catheter body 202 to be used without a second or outflow orifice (e.g., positioned proximally of the distal end 208). Alternatively, some of the entrapped thrombogenic material may exit the catheter body 202 through a second orifice (not shown), e.g., in a sidewall of the catheter body 202, positioned proximal to the distal end 208, recirculate to the distal end 208 (e.g., one or more times) or the funnel 230, and then move proximally through the lumen 206 of the catheter body 202.

In some examples, the supply tube 210 may be designed to translate longitudinally relative to the catheter body 202 and the funnel 230. Further, the supply tube 210 may be designed to translate longitudinally relative to the catheter body 202 and the funnel 230 such that the distally projecting orifice 218c, the proximally projecting orifice 218b or both the distally projecting orifice 218c and the proximally projecting orifice 218b extend distal of the distal end 208 of the catheter body 202. For example, FIG. 6 illustrates that supply tube 210 after having been translated in a proximal-to-distal direction relative to the distal end 208 of the catheter body 202 such that the distally projecting orifice 218c and the proximally projecting orifice 218b extend a distance “Z” distal to the distal end 208 of the catheter body 202. In other words, FIG. 6 illustrates that supply tube 210 after having been translated in a proximal-to-distal direction relative to the distal end 208 of the catheter body 202 such that the distally projecting orifice 218c and the proximally projecting orifice 218b extend a distance Z into the funnel 230. In some examples, the distance Z that the distal end 216 of the supply tube 210 extends distally past the distal end 208 of the catheter body 202 (e.g., the distance Z the distal end 216 extends into the funnel 230) may be about 0 mm to 40 mm, or about 5 mm to about 35 mm, or about 10 mm to about 30 mm, or about 15 mm to about 25 mm, or about 20 mm.

In some examples, the supply tube 210 may move longitudinally (e.g., axially) between a first position in which the distal end 216 of the supply tube 210 is positioned proximal of the distal end 216 of the catheter body 202 and a second position in which the distal end 216 of the supply tube 210 is positioned distal of the distal end 216 of the catheter body 202. Further, it can be appreciated that supply tube 210 may oscillate between the proximal position and the distal position in conjunction with a pumping cycle of the pump 56 of the thrombectomy catheter. For example, the pumping cycle of the pump 56 may include a down stroke and an upstroke. The supply tube 210 may shift from a proximal position (whereby distal end 216 of the supply tube 210 is positioned proximal of the distal end 216 of the catheter body 202) to the distal position (whereby the distal end 216 of the supply tube 210 is positioned distal of the distal end 216 of the catheter body 202) during the downstroke of the pumping cycle. Additionally, the supply tube 210 may shift from a distal position (whereby distal end 216 of the supply tube 210 is positioned distal of the distal end 216 of the catheter body 202) to the proximal position (whereby the distal end 216 of the supply tube 210 is positioned proximal of the distal end 216 of the catheter body 202) during the upstroke of the pumping cycle. The supply tube 210 may oscillate between these two positions as the pump cycles between the downstroke and the upstroke.

As discussed herein, a proximal end of the high-pressure fluid supply tube 210 may be in fluid communication with the pump 56 (shown in FIG. 1), to provide high-pressure fluid to the fluid pathway 214 of the high-pressure fluid supply tube 210. The high-pressure fluid passing through the fluid supply tube 210 may contact thrombogenic material positioned at the distal end region of the funnel 230. The distally projecting jet orifice 218c may macerate any thrombogenic material that enters the distal opening of the funnel 230 thus helping prevent clogging. For example, at the point of impingement of the distally oriented motive jetted fluid 220c the motive jetted fluid 220c may deflect distally creating flow out the funnel 230, effectively macerating any clot that enters the distal end of the device and eliminating or reducing risk of the funnel 230 becoming blocked or clogged. It is contemplated that the properties (size, shape, angle, number, spacing, etc.) of the jet orifices 218 may be varied to obtain a fluid velocity that creates an optimum de-clogging effect without hindering the proximal flow of a clot within the funnel 230 and/or catheter body 202. It can be further appreciated that the oscillating movement of the supply tube 210 (e.g., mechanical probing/jabbing) may fracture the thrombogenic material. Additionally, it can be further appreciated that the oscillating movement (e.g., mechanical probing/jabbing) of the supply tube 210 may aid in the maceration of any thrombogenic material entering the funnel 230.

FIG. 7 illustrates another embodiment of the thrombectomy catheter 200. FIG. 7 illustrates that, in some examples, the supply tube 210 may include only a single distally projecting jet orifice 218c.

FIG. 8 illustrates that, in some examples, supply tube 210 may include one or more projections 240 positioned along the distal end region of the supply tube 210. For example, FIG. 8 illustrates that the supply tube 210 may include one or more projections 240 attached to the distal facing surface 216 of the supply tube 210. Each of the projections 240 may extend in a distal direction from the distal facing surface 216. In some examples, the one or more projections may include blades, hooks, J-hooks, barbs, prong, quill, spike, spur, arrow or any combination thereof.

It can be appreciated that the one or more projections positioned on the distal end region of the supply tube 210 may aid the oscillating supply tube in breaking thrombogenic material that may enter or be adjacent to the funnel 230. In other words, as the supply tube 210 oscillates during the pumping cycle, the one or more projections may repeatedly stab and be reburied in the thrombogenic material, thereby helping to break up the clot.

FIGS. 9-12 illustrates an example method of the thrombectomy catheter 200 being utilized to treat a target treatment site 244 (e.g., clot, thrombus) positioned in a body vessel 250.

FIG. 9 illustrates that the thrombectomy catheter 200 (of the example thrombectomy system 10 described herein) may be delivered to a treatment site through a tubular delivery shaft 242. As described herein, when being tracked to the treatment site 244, the funnel 230 may be in a collapsed and loaded configuration, whereby the delivery shaft 242 may be tracked through the patient's body vessel 250 and positioned adjacent to a target treatment site 244 (e.g., thrombus). It can be appreciated from the discussion herein that the supply tube 210 and the catheter body 202 are also positioned within the tubular delivery shaft 242 shown in FIG. 9.

FIG. 10 illustrates that after being positioned adjacent to the target treatment site 244, the delivery shaft 242 may be retracted in a distal-to-proximal direction. It can be appreciated that upon proximal retraction of the delivery shaft 242, the self-expanding funnel 230 may shift from the unemployed, delivery configuration to the deployed configuration. FIG. 10 also illustrates a portion of the supply tube 210 being uncovered as the delivery shaft 242 is retracted proximally.

As discussed herein, when in the deployed configuration, the funnel 230 may be designed to expand such that the distal end 234 of the funnel 230 may engage the inner surface 236 of a body vessel 250 within which it is deployed. In some instances, the delivery shaft 242 may be positioned such that when it is retracted in a distal-to-proximal direction, the distal end 234 of the funnel contacts the thrombus 244. In some instances, the funnel 230 may be expanded in a position such that the entire circumference of the distal end 234 of the funnel 234 may contact the thrombus 244. Deploying the funnel 230 in this configuration may help prevent blood loss during the thrombectomy procedure.

FIGS. 11-12 illustrate the oscillating action of the supply tube 210 as the pump 56 of the thrombectomy system 10 is operated to infuse fluid 220a-c (e.g., a motive fluid, a liquid, a gas or air, steam, a fluid with particles disposed therein, or the like) through the jet orifices and into the lumen 206 of the catheter body 202 and/or the funnel 230. For example, FIG. 11 illustrates the supply tube 210 in a first position in which the distal end region of the supply tube 210 (including the projections 240 described herein) are proximal of the distal end 208 of the catheter body 202. As discussed herein, FIG. 12 illustrates the supply tube 210 in a second position in which the distal end region of the supply tube 210 (including the projections 240 described herein) are distal of the distal end 208 of the catheter body 202. Both FIGS. 11-12 illustrates that jet orifice 218c projecting a motive fluid in a generally distal direction as depicted by lines 220c and jet orifices 218a-b projecting a motive fluid in a proximal direction as depicted by lines 220a-b. FIGS. 11-12 illustrate the repeated jabbing action of the projections 240 to break up the thrombus 244 into smaller pieces 246, which are pulled into the funnel 230 and through the lumen 206 of the supply tube 210 to a location outside the patient's body.

The materials that can be used for the various components of the thrombectomy catheter, pump/catheter assembly, and/or other devices disclosed herein may include those commonly associated with medical devices. For simplicity purposes, the following discussion refers to the pump/catheter assembly and its related components. However, this is not intended to limit the devices and methods described herein, as the discussion may be applied to other similar devices, tubular members and/or components of tubular members or devices disclosed herein.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. An aspiration catheter, comprising:
a catheter body having a proximal end region, a distal end region and a lumen extending therein;
a fluid supply tube extending within the lumen of the catheter body, the fluid supply tube having a distal end region and a distal facing surface; and
a funnel having a proximal end and a distal end, wherein the proximal end is coupled to the distal end region of the catheter body, and wherein the funnel is configured to shift between a collapsed configuration and an expanded configuration.

2. The aspiration catheter of claim 1, wherein the fluid supply tube is configured to shift between a first position in which the distal end region is positioned proximal of the proximal end of the funnel and a second position in which the distal end region is positioned distal of the proximal end of the funnel.

3. The aspiration catheter of claim 1, wherein the fluid supply tube is configured to shift between a first position in which the distal end region is positioned proximal of the proximal end of the funnel and a second position in which the distal end region is positioned between the distal end and the proximal end of the funnel.

4. The aspiration catheter of claim 2, wherein the proximal end region of the catheter body is coupled to a fluid pump, and wherein the fluid pump is configured to cycle between applying a vacuum and ceasing to apply the vacuum, and wherein the fluid supply tube shifts between the first position and the second position based on the vacuum cycle of the fluid pump.

5. The aspiration catheter of claim 4, wherein the fluid supply tube is configured to shift from a first position in which the distal end region is positioned proximal of the proximal end of the funnel and a second position in which the distal end region is positioned between the distal end and the proximal end of the funnel when the fluid pump is in a downstroke, and wherein the fluid supply tube is configured to shift from the second position to the first position when the fluid pump is in an upstroke.

6. The aspiration catheter of claim 2, wherein when the fluid supply tube is in the second position, the distal facing surface is between 0 mm and 20 mm distal of the proximal end of the funnel.

7. The aspiration catheter of claim 1, wherein when in the expanded configuration the funnel tapers from a first diameter at its proximal end to a second diameter at its distal end, and wherein the second diameter is greater than the first diameter.

8. The aspiration catheter of claim 1, wherein the funnel includes a plurality of braided filaments.

9. The aspiration catheter of claim 1, wherein funnel includes at least one reinforcing member, and wherein the at least one reinforcing member extends from a proximal end of the funnel to a distal end of the funnel.

10. The aspiration catheter of claim 1, wherein the funnel is configured to direct thrombus into the lumen of the catheter shaft.

11. The aspiration catheter of claim 1, wherein when in the expanded configuration the distal end region of the funnel is configured to engage an inner surface of a body vessel.

12. The aspiration catheter of claim 1, further comprising at least one projection extending distally away from the distal facing surface of the fluid supply tube.

13. The aspiration catheter of claim 1, wherein the fluid supply tube includes at least one distally projecting jet orifice for expelling at least one distally oriented fluid jet in a generally distal direction from the fluid supply tube within the catheter lumen or funnel.

14. The aspiration catheter of claim 13, wherein the fluid supply tube includes at least one proximally projecting jet orifice for expelling at least one proximally oriented fluid jet in a generally proximal direction from the fluid supply tube within the catheter lumen or funnel.

15. The aspiration catheter of claim 14, wherein the at least one distally projecting jet orifice is distal to the at least one proximally projecting jet orifice.

16. The aspiration catheter of claim 13, wherein the at least one distally projecting jet orifice extends through a sidewall of the fluid supply tube.

17. A thrombectomy system, comprising:
a processor coupled to a fluid pump and a thrombectomy catheter, wherein the thrombectomy catheter includes:
a catheter body having a proximal end region, a distal end region and a lumen extending therein;
a fluid supply tube extending within the lumen of the catheter body, the fluid supply tube having a distal end region and a distal facing surface; and
a funnel having a proximal end and a distal end, wherein the proximal end is coupled to the distal end region of the catheter body, and wherein the funnel is configured to shift between a collapsed configuration and an expanded configuration.

18. The aspiration catheter of claim 17 wherein the fluid supply tube is configured to shift between a first position in which the distal end region is positioned proximal of the proximal end of the funnel and a second position in which the distal end region is positioned distal of the proximal end of the funnel.

19. The aspiration catheter of claim 18, wherein the proximal end region of the catheter body is coupled to a fluid pump, and wherein the fluid pump is configured to cycle between applying a vacuum and ceasing to apply the vacuum, and wherein the fluid supply tube shifts between the first position and the second position based on the vacuum cycle of the fluid pump.

20. A thrombectomy system, comprising:
a processor coupled to a fluid pump and a thrombectomy catheter, wherein the thrombectomy catheter includes:
a catheter body having a proximal end region, a distal end region and a lumen extending therein;
a fluid supply tube extending within the lumen of the catheter body, the fluid supply tube having a distal end region and a distal facing surface; and
a funnel having a proximal end and a distal end, wherein the proximal end is coupled to the distal end region of the catheter body, and wherein the funnel is configured to shift between a collapsed configuration and an expanded configuration;
wherein the fluid supply tube is configured to shift between a first position in which the distal end region is positioned proximal of the proximal end of the funnel and a second position in which the distal end region is positioned distal of the proximal end of the funnel;
wherein the fluid pump is configured to cycle between applying a vacuum and ceasing to apply the vacuum, and wherein the processor is configured to direct the fluid supply tube to shift between the first position and the second position based on the vacuum cycle of the fluid pump.
